(19) United States
(12) Patent Application Publication
(10) Pub. No.: US 2015/0342521 A1
(43) Pub. Date: Dec. 3, 2015

(54) SURGICAL APPARATUS AND SYSTEM

(71) Applicant: ASSOCIATION OF NANTAN GENERAL HOSPITAL FOR NATIONAL HEALTH INSURANCE, Kyoto (JP)

(72) Inventors: Wataru NARITA, Kyoto (JP); Taku OGURA, Kyoto (JP); Ryota TAKATORI, Kyoto (JP); Toshikazu KUBO, Kyoto (JP)

(21) Appl. No.: 14/537,432

(22) Filed: Nov. 10, 2014

(30) Foreign Application Priority Data
May 29, 2014 (JP) 2014-111582

(51) Int. Cl. A61B 5/00; A61B 5/0492

(52) U.S. Cl. CPC A61B 5/4893; A61B 5/0492; A61B 5/6826

(57) ABSTRACT

An apparatus for monitoring at or near a nerve during patient surgery, the apparatus being connectable to a nerve monitoring system, comprises: a finger attachment portion; a connecting portion connectable to the nerve monitoring system; a cable for connecting the connecting portion to the finger attachment portion; and an electrode provided in the finger attachment portion.

### CROSS REFERENCE TO RELATED APPLICATIONS

This application is based upon and claims the benefit of priority from Japanese Patent Application No. 2014-111582, filed on May 29, 2014, the entire contents of which are incorporated herein by reference.

### STATEMENT RE: FEDERALLY SPONSORED RESEARCH/DEVELOPMENT

Not Applicable.

### BACKGROUND OF THE INVENTION

This invention relates to a surgical apparatus and a system. More particularly, this invention relates to a surgical apparatus and a system for establishing a passage for inserting a surgical instrument by moving aside, with a finger, adipose tissue and muscle in the proximity of a nerve.

In various surgical operations, it is necessary to secure a passage for a surgical instrument to access a target site. It is possible that such passages will be located near nerves. For example, in spinal surgery, such as intervertebral disc surgery, a passage for the surgical instrument must be secured by moving aside adipose tissue and/or iliopsoas muscle.

WO2004/064634 discloses a system for establishing an operative corridor using a rod-like surgical accessory with a sensor for detecting a distance from a nerve; the surgical accessory is inserted from a dissected portion of a skin toward the spine. In this system, the position of the surgical accessory is monitored with electromyography (EMG). However, subtle positioning between the surgical accessory and the nerve is difficult, and the skill of an experienced doctor was necessary to secure the operative corridor without injuring the lumbar plexus. In addition, after the operative corridor for a spine instrument is secured, the position of the spine instrument and the spine can be monitored by X-ray fluoroscope and a navigation system. However, when the operative corridor is not secured sufficiently, the spine operation does not proceed smoothly.

### SUMMARY OF THE INVENTION

An object of the invention is to provide an apparatus and a system for facilitating a surgical operation by moving aside muscle and/or adipose tissue near a nerve more safely and securely.

The invention provides the following apparatus and systems.

In one aspect of the invention, an apparatus for monitoring at or near a nerve during patient surgery, the apparatus being connectable to a nerve monitoring system, comprises: a finger attachment portion; a connecting portion connectable to the nerve monitoring system; a cable for connecting the connecting portion to the finger attachment portion; and an electrode provided in the finger attachment portion.

In one embodiment, the finger attachment portion is made of flexible silicone rubber or flexible plastic.

In another embodiment, the apparatus is made of a sterilizable material.

In yet another embodiment, the apparatus further comprises a tube for covering the cable and connecting the connecting portion with the finger attachment portion.

In a second aspect of the invention, a surgical nerve location detecting system comprises a nerve monitoring system and an apparatus. The apparatus includes a finger attachment portion, a connecting portion connectable with a nerve monitoring system, a cable for connecting the connecting portion with the finger attachment portion, and an electrode provided in the finger attachment portion. The nerve monitoring system includes a body surface electrode, and a control unit for electromyography (EMG) measurement between the body surface electrode and the electrode provided in the finger attachment portion to determine a current threshold at which the muscle reacts, so as to detect the location of a nerve.

In one embodiment, the finger attachment portion is made of flexible silicone rubber or flexible plastic.

In another embodiment, the apparatus is made of a sterilizable material.

In yet another embodiment, the apparatus further includes a tube for covering the cable and connecting the connecting portion to the finger attachment portion.

In a third aspect of the invention, a method for detecting the location of a nerve with an apparatus connected to a nerve monitoring system, comprises: positioning a body surface electrode on a patient; inserting the apparatus in to a patient, wherein the apparatus includes a finger attachment portion, a connecting portion connectable with a nerve monitoring system, a cable for connecting the connecting portion to the finger attachment portion, and an electrode provided in the finger attachment portion; and detecting the location of a nerve by electromyography (EMG) measurement between the body surface electrode and the electrode provided in the finger attachment portion to determine a current threshold at which the muscle reacts so as to detect the location of a nerve.

### BRIEF DESCRIPTION OF THE DRAWINGS

FIG. 1 is a schematic view of an apparatus according to the first embodiment of the invention.

FIG. 2 is a view of the apparatus of FIG. 1 attached to the finger.

FIG. 3 is a schematic view of a surgical nerve location detecting system including the apparatus of FIG. 1.

FIGS. 4A to 4C are schematic views in which the apparatus of the invention is inserted toward the spine, after which an instrument for excising a portion of an intervertebral disc is inserted.

FIG. 5 is a schematic view of an apparatus according to another embodiment of the invention.

### DETAILED DESCRIPTION OF THE INVENTION

In the specification, the terms “a,” “an,” “the,” and similar referents in the context of describing the invention should be construed as including both the singular and the plural, unless otherwise indicated herein or clearly inconsistent with the context.

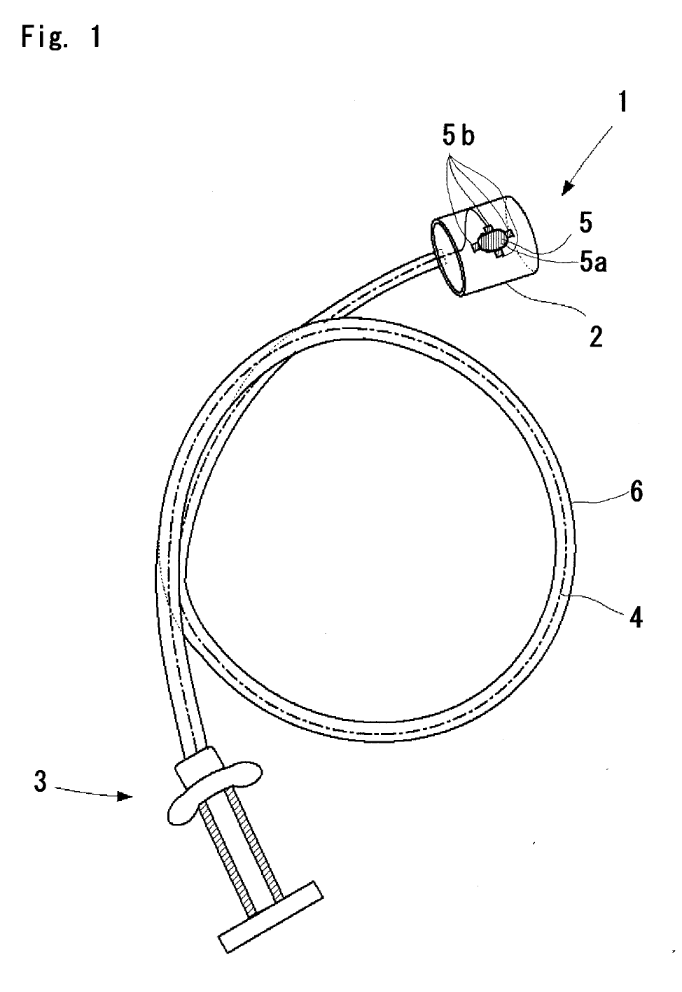

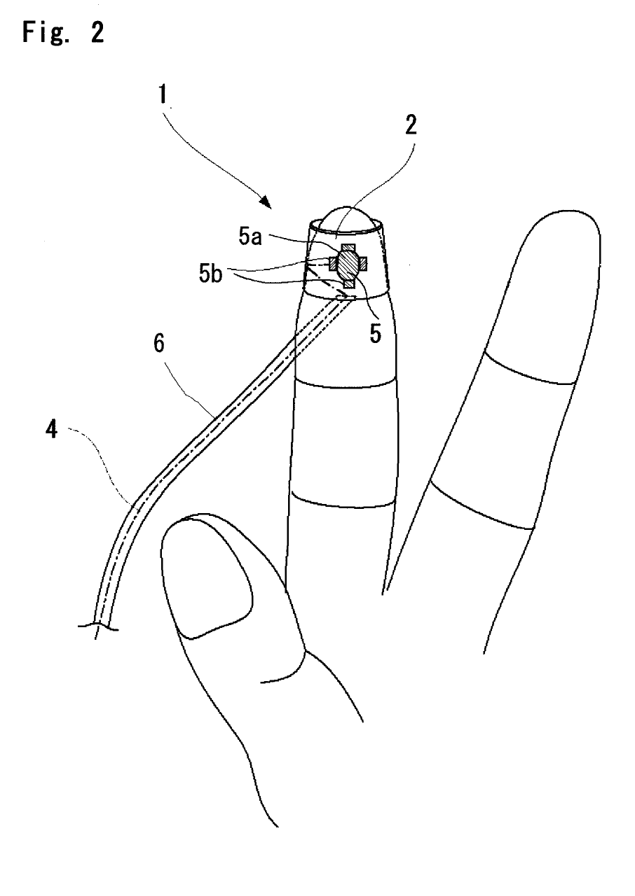

The first embodiment of the apparatus of the invention is illustrated in FIGS. 1 and 2.

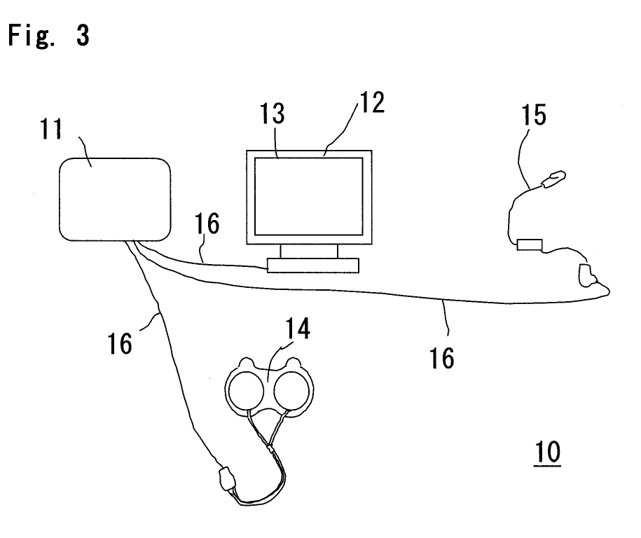

Referring to FIG. 1, an apparatus 1 of the invention includes a finger attachment portion 2, a connecting portion 3 connectable to a nerve monitoring system 10 (see FIG. 3), an electrically conductive cable 4 that connects the connecting portion 3 to the finger attachment portion 2, and an electrode 5 provided in the finger attachment portion 2. The finger attachment portion 2 is in the form of a fingerstall. Specifically, the finger attachment portion 2 is annularly shaped to fit around the finger, and is fitly attached to the finger. Accordingly, the finger attachment portion 2 remains attached to the finger, even when the finger is moved in tissue such as adipose tissue and muscle, or in a body fluid such as blood and tissue fluid; moreover, the position of the electrode 5 remains fixed. The finger attachment portion 2 may be formed of any medical material. The finger attachment portion 2 is preferably made of a flexible material, more preferably silicone rubber or plastic. The electrode 5 is typically made of metal. The electrode 5 is disposed within the finger attachment portion 2, or attached to the finger attachment portion 2; accordingly, the electrode 5 is isolated from the doctor's finger to which the finger attachment portion 2 is attached. In FIG. 1, the electrode 5 includes a generally semi-ellipsoid main portion 5a that is disposed within the finger attachment portion 2 and that convexes radially outward from the finger attachment portion 2, and four extended portions 5b that are disposed perpendicular to each other (in a relation such that two straight lines that extend through the two adjacent electrodes 5 are perpendicular). A portion of the connecting portion 3 that is connected to the nerve monitoring system 10 is made of an electrically conductive material. The connecting portion 3 can be connected to the nerve monitoring system 10 by, for example, a clip 15 (see FIG. 3) that pinches the connecting portion 3. The distal end of the cable 4 is connected to one of the extended portions 5b of the electrode 5. The proximal end of the tube 4 is connected to the connecting portion 3. The cable 4 may be covered with an insulative sheath or tube 6. The tube 6 may be formed of any medical material. The tube 6 is preferably made of a flexible material, more preferably silicone rubber or plastic. The finger attachment portion 2 and the tube 6 may be formed of the same material, or different materials. The distal end of the tube 6 is connected to the finger attachment portion 2 at a position different from that of the electrode 5 so as not to prevent movement of the finger when a doctor moves his or her finger with the finger attachment portion 2 attached thereto. Preferably, the distal end of the tube 6 is connected to the finger attachment portion 2 on the opposite side from the electrode 5 in a direction circumferential to the finger attachment portion 2. The proximal end of the tube 6 is connected to the connecting portion 3.

Referring to FIG. 2, when the apparatus 1 of the invention is attached to a doctor's finger, the finger attachment portion 2 is attached in such a manner that the electrode 5 is located on the ball of the finger (palm side), and the cable 4 is located on the nail side of the finger. Since the tube 6 having the cable 4 and the finger attachment portion 2 are made of a flexible material, these members do not interfere with the movement of the finger. Since the tube 6 and the finger attachment portion 2 are inserted into the body, by removably connecting the tube 6 with the connecting portion 3, the tube 6, the cable 4, and the finger attachment portion 2 can be made disposable. Further, since finger size differs depending on the doctor, by making the cable 4 removable from the connecting portion 3 or additionally making the finger attachment portion 2 removable from the tube 6 and the cable 4, a finger attachment portion 2 that suits the size of the finger of the doctor conducting the operation can be selected.

The electrode 5 is configured so that a threshold (a current value at or above that by which the muscle reacts) is displayed on a monitor 13 of a display unit 12 of the nerve monitoring system 10 by electromyography (EMG) measurement between the body surface electrode 14 provided on each muscle of the lower leg and the finger attachment portion 2. When the threshold of the electromyogram is low, the distance between the electrode 5 and the nerve is short. When the threshold is high, the distance between the electrode 5 and the nerve is long. The measurement of the distance from the nerve by the threshold of the electromyogram is described in WO2004/064634.

Referring to FIG. 3, the nerve monitoring system 10 includes a control unit 11, the display unit 12 having the monitor 13, the body surface electrode 14, the clip 15, and leads 16 that connect the members 11, 12, 14, and 15. The threshold of the electromyogram is determined by the control unit 11, and displayed at the display unit 12 depending on the distance from the nerve. As such a nerve monitoring system, an NVM5 (registered trademark) nerve monitoring system, produced by NuVasive, Inc., is known. Details regarding this system are also described in WO2004/064634, which is incorporated herein by reference.

The threshold can be measured when the electrode 5 faces in the direction of the nerve when the finger attachment portion 2 is attached to the doctor's finger. However, the threshold cannot be measured when the electrode 5 does not face the nerve. This enables confirmation of the direction of the nerve with the finger. The electrode 5 can stimulate the nerve with a current pulse. Since the nerve can or cannot be detected depending on the change in the direction of the electrode 5 when the finger is rotated, the doctor conducting the operation can confirm the direction of the nerve. In addition, since the current value of the threshold changes when the location of the electrode 5 is moved as the finger moves, the doctor can confirm the distance between the nerve and the finger. Since the direction and the distance from the nerve changes depending on the current amount of the threshold, by setting an appropriate amount of the current pulse, the positional information of the nerve in the direction and the distance in the range required for the operation can be obtained. When adipose tissue and/or muscle (the psoas major) near the spine is moved aside, the manipulation at the time of the operation can be appropriately performed by getting the doctor's attention by displaying an image, emitting a warning sound, or blinking a red light when there is possibility of nerve injury when the nerve is facing and near the electrode 5, and the current amount of the threshold is small.

Indication means in the display unit 12 include an indication with an image, a voice, a sound, light, and vibration. Specifically, the indication means is a monitor 13 when an image is indicated. The indication means is a speaker when a sound is indicated. The indication means also include a sound source tip (for example, a sound source such as a CPU). The indication means is a light source when light is indicated. The indication means is a vibration source when a vibration is indicated. For example, two or three indications, such as an image and a voice/sound/light, may be combined to show a doctor that the distance from the nerve is short.

When a voice/sound, light, heat, or vibration is indicated, a louder voice/sound, stronger light, stronger vibration, and higher heat may be generated for the indication as the nerve becomes closer. When an image is indicated, the color may be changed. For example, red is indicated when the nerve is close, and yellow and blue are indicated as the nerve becomes farther away. The control unit 11 can be set so that nothing is indicated when the distance between the nerve and the electrode 5 is the same or shorter than a predetermined value.

Since the apparatus 1 of the invention is used while it is attached to the finger, minor adjustment with the finger movement is possible compared to the case when the monitor 13 and the apparatus 1 are used for remote manipulation. Thus, the position of the adipose tissue and the muscle tissue around the nerve can be readily moved without injuring the nerve. A passage for inserting a surgical instrument is secured, and a subsequent spinal cord operation can be performed smoothly.

A spine lateral approach is included as an example of a spinal cord operation. Specifically, excision of an intervertebral disc and insertion of a plate are included. Although the spine lateral approach is preferred, the apparatus 1 and a system of the invention may also be used in an anterior or posterior approach of the spine. Although use in a spinal cord operation is described in this embodiment, it is understood that the apparatus 1 and the nerve location detecting system of the invention are also suitable for use in surgery on other parts of the body where the apparatus of the invention must pass at or near nerve tissue, such as peripheral nerves of limbs or cranial nerve, to secure passage to a surgical operation site.

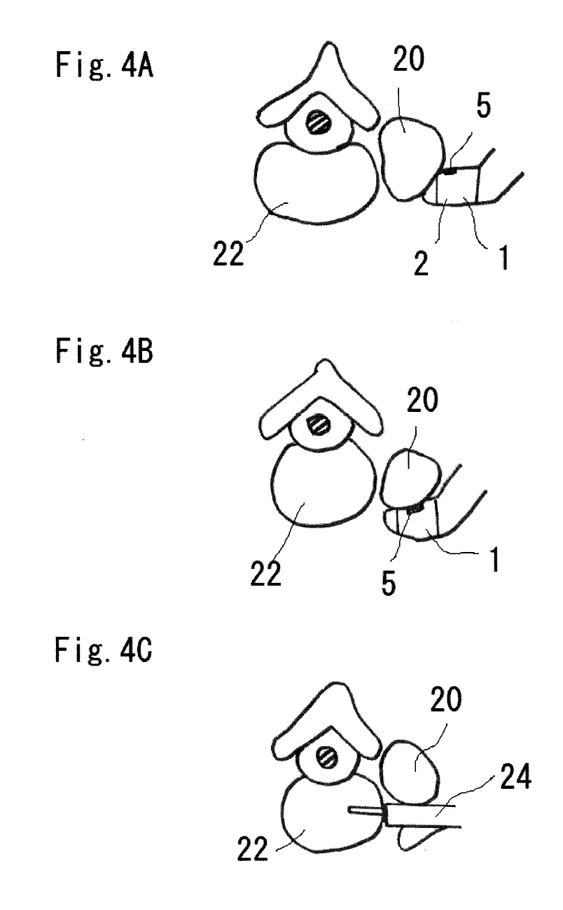

As illustrated in FIGS. 4A to 4C, the doctor's finger to which the finger attachment portion 2 is attached can be inserted from an incision of the skin on the side of the upper body of the patient toward the spine. In a situation where the adipose tissue or major psoas 20 is moved aside using the apparatus 1 of the invention, an instrument 24 for excising a portion of the intervertebral disc 22 is inserted from the incision into the body to exercise the intervertebral disc and to perform plate insertion. As such an instrument for excising the intervertebral disc, a CoRoent® system titanium cage X/X Plus, a CoRoent XLIF operation machine, a Maxcess operation set, and an XLP spine plate system by NuVasive, Inc. can be used.

Each nerve, including the nerves around the spine, has a current of characteristic threshold level. A current stimulation that is below the threshold of the electrode does not induce a notable nerve response. Once the stimulus threshold is reached, the induced response becomes reproducible, and increases to the limit as the stimulation increases. The current pulse at the electrode is set to a predetermined value in the range that causes the nerve response.

According to the invention, surgical operation can be performed without injuring a nerve by confirming the location of the nerve with a finger, and moving adipose tissue and/or muscle around the operation site aside. Thus, even a less-skilled doctor can perform the operation smoothly.

While the invention has been described by exemplifying the first embodiment, the invention is not limited thereto, and may include various modifications within the scope and equivalence of the appended claims as follows.

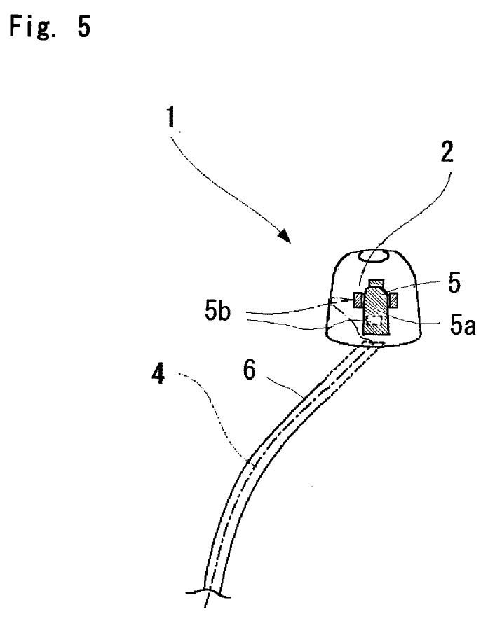

As illustrated in FIG. 5, the diameter of the distal portion of the finger attachment portion 2 may be reduced to fit the fingertip. In addition, the main body 5a of the electrode 5 may be extended in one direction to enhance sensitivity by increasing a contact area between the electrode 4 and a region of a patient. Alternatively or additionally, the main body 5a of the electrode 5 may be omitted, or the electrode 5 may take any other form as long as the current threshold can be measured. The form and the number of the extended portions 5b are not limited to those of the first embodiment.

What is claimed is:

1. An apparatus for monitoring at or near a nerve during patient surgery, the apparatus being connectable to a nerve monitoring system, comprising:
a finger attachment portion;
a connecting portion connectable to the nerve monitoring system;
a cable for connecting the connecting portion to the finger attachment portion; and
an electrode provided in the finger attachment portion.

2. The apparatus of claim 1, wherein the finger attachment portion is made of flexible silicone rubber or flexible plastic.

3. The apparatus of claim 1, wherein the apparatus is made of a sterilizable material.

4. The apparatus of claim 1, further comprising a tube for covering the cable and connecting the connecting portion with the finger attachment portion.

5. A surgical nerve location detecting system comprising:
a nerve monitoring system; and
an apparatus including a finger attachment portion, a connecting portion connectable with a nerve monitoring system, a cable for connecting the connecting portion with the finger attachment portion and an electrode provided in the finger attachment portion, wherein the nerve monitoring system includes a body surface electrode; and
a control unit for electromyography (EMG) measurement between the body surface electrode and the electrode provided in the finger attachment portion to determine a current threshold at which the muscle reacts so as to detect the location of a nerve.

6. The nerve location detecting system according to claim 5, wherein the finger attachment portion is made of flexible silicone rubber or flexible plastic.

7. The nerve location detecting system according to claim 5, wherein the apparatus is made of a sterilizable material.

8. The nerve location detecting system according to claim 5, the apparatus further including a tube for covering the cable and connecting the connecting portion with the finger attachment portion.

9. A method for detecting the location of a nerve with an apparatus connected to a nerve monitoring system, comprising:
positioning a body surface electrode on a patient;
inserting the apparatus into a patient, wherein the apparatus includes a finger attachment portion, a connecting portion connectable to a nerve monitoring system, a cable for connecting the connecting portion to the finger attachment portion and an electrode provided in the finger attachment portion; and
detecting the location of a nerve by electromyography (EMG) measurement between the body surface electrode and the electrode provided in the finger attachment portion to determine a current threshold at which the muscle reacts so as to detect the location of a nerve.
